(19) United States
(12) Patent Application Publication
(10) Pub. No.: US 2010/0099169 A1
(43) Pub. Date: Apr. 22, 2010

(54) GENETIC SELECTION SYSTEM FOR IMPROVING RECOMBINANT PROTEIN EXPRESSION

(76) Inventors: James U. Bowie, Culver City, CA (US); Elizabeth M. Gendel, Los Angeles, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/579,298

(22) Filed: Oct. 14, 2009

Related U.S. Application Data
(60) Provisional application No. 61/107,626, filed on Oct. 22, 2008.

(51) Int. Cl. C12N 1/21

(52) U.S. Cl. 435/252.33

(57) ABSTRACT

A method for selecting host cells with an improved ability to recombinantly overexpress a target protein; the host cells thus generated and their use. The invention also provides a curing method to remove plasmids from host cell lines.

### BACKGROUND

The present invention relates to a genetic selection system which is used to generate novel host cells with an improved ability to overexpress a target protein, and to the host cells thus generated and their use in expression of polypeptides.

Microorganisms, and especially bacteria such as Escherichia coli, are among the most successful vehicles for over-expression of both prokaryotic and eukaryotic proteins (Hockney, 1994; Grisshammer & Tate, 1995; Terpe, 2006). Many different expression systems are known in the art for the expression of both endogenous and foreign proteins. In these expression systems, DNA encoding the target protein of interest is encoded on an expression vector, and the total coding sequence is operably linked to a promoter such that the promoter drives expression of the coding sequence.

Such expression systems employed to over-express proteins, however, are not always satisfactory. Some proteins, for instance, cannot be produced in sufficient quantities for functional and structural studies or alternatively for commercial production on an industrial scale. Furthermore, some proteins that can be expressed at high levels are expressed as insoluble inclusion bodies that cannot be refolded functionally or that cannot be refolded at all. Regardless, even if a target protein can be refolded functionally to do so would be inefficient and costly on an industrial scale. Protein targets that are difficult to overexpress include targets of both prokaryotic and eukaryotic origin and both membrane and globular proteins.

In the case of membrane proteins, overexpression is especially challenging and presents a major barrier to the biochemical, physical, and structural characterization of many membrane proteins. This problem is illustrated in a study by Korepanova et al. wherein the Mycobacterium tuberculosis (MTb) alpha-helical membrane proteome was expression-tested in various E. coli strains (2005). Out of the 105 membrane protein targets tested, only 37 were over-expressed sufficiently to be detectable by Coomassie staining. Only 9 of those 37, all less than 16 kD, were expressed to the membrane fraction and presumably correctly folded. Thus, the standard expression techniques used in this study resulted in high-level expression to the membrane for only 10% of the targets, a success rate that is not unusual for membrane proteins (Lewinson et al. 2008).

Nonetheless, because E. coli is such an extremely convenient host it is desirable to increase the success rate of E. coli expression to produce protein for biochemical and structural studies and as a host to produce protein on an industrial scale. Bacteria such as E. coli are the first choice for functional and structural studies, and bacterial expression systems are used in industry to express a wide variety of proteins, including chymosin, insulin, interferons, insulin-like growth factors, antibodies including humanized antibodies, or fragments thereof (Farid, 2006; Graumann and Premstaller, 2006). Given the widespread use of microorganisms for polypeptide expression in both research and industry settings, there is a continuing need for improved expression systems.

There are myriad reasons why expression systems fail. Potential problems common to all heterologous protein expression include codon usage, mRNA or protein stability, and cell physiology changes induced by the stress of recombinant expression (Sorensen and Mortensen 2005). With membrane protein production, there are numerous additional possible failure points because insertion requires proper targeting of the nascent polypeptide chain and intimate interactions with the insertion machinery (Grisshammer, 2006). These complexities are well illustrated by an impressive proteomics study of the effects of membrane protein expression in E. coli by Wagner et al. (2007). Among many changes, they found alterations in chaperone machinery, evidence of energy stress, and impairment of native membrane protein as well as secretory protein expression. Thus, membrane protein expression can have dramatic consequences for many aspects of cell physiology. It remains unclear, however, what the critical factors are that limit proper expression and insertion, and the key barriers may be different for each membrane protein.

Current approaches to improving expression generally employed in an ad hoc fashion, including altering growth media, temperature, or induction levels. In addition, fusion to other proteins can help expression. Fusion to Mistic appears to be particularly helpful and is thought to help chaperone the protein into the membrane (Roosild et al. 2005). Another approach is cell-free expression which can bypass deleterious changes in cell physiology (Klammt et al. 2007). The produced protein, however, is not necessarily in a folded, functional form, and this is particularly troublesome for membrane proteins, which tend to be difficult to re-fold. In other studies, the target protein is mutated to obtain a more stable variant that can be successfully expressed, but this is undesirable in that the expressed mutant may not retain wild type structure and function (Martinez Molina et al., 2008; Sarkar et al., 2008). Although these techniques can offer significant improvement in isolated cases, there have been no universal solutions and protein production continues to be a considerable obstacle that must be addressed.

Because of the many possible points in protein biogenesis that could go awry and prevent expression, a rational, hypothesis-driven approach to the problem is difficult. An alternative approach is to select for genomic mutations that improve expression, obviating the need for a precise understanding of the barriers to protein production. Along these lines, Miroux and Walker isolated strains of E. coli that were resistant to the toxicity of membrane protein expression (1996). Accordingly, their invention (U.S. Pat. No. 6,361,966) provides a method for improving an expression system comprising the steps of: (a) preparing an expression system consisting essentially of a host cell transformed with an inducible expression vector encoding a target polypeptide and a selectable marker; (b) culturing cells transformed with the expression system under selection pressure for plasmid maintenance; (c) inducing the expression system to produce the target polypeptide, such that a toxic effect is observable in the host; (d) recovering host cells from the culture and growing them under a selection pressure and inducing conditions; and (e) selecting viable host cells which continue to produce the target polypeptide. Mutant hosts which have evolved a resistance to system toxicity and retain the ability to express the target gene from the expression vector appear as small colonies on agar plates, in contrast to large colonies, which have lost the ability to express the target gene or that have lost the vector and have derived an alternative antibiotic resistance means in order to survive. The strains isolated by Miroux and Walker, called C41(DE3) and C43(DE3), improve expression of many membrane proteins expressed from the T7 promoter and are now routinely used in expression screening. These mutant strains were found to act by slowing expression from the strong T7 promoter (Wagner et al., 2008). These results clearly indicate that it is possible to re-engineer E. coli with an improved ability to express membrane proteins.

The techniques used by Miroux and Walker to isolate C41 and C43, however, are limiting in several respects. Firstly, in their method it is necessary that overexpression of the target protein result in death of the wild type host cell line. Not all expression systems and not all poorly expressed proteins, however, are toxic to the point of killing host cells, and so the method of Miroux and Walker is limited to a subset of proteins and expression systems that are extremely toxic. Even when applied to extremely toxic proteins, the invention of Miroux and Walker is limiting in another respect in that toxicity can be eliminated in ways that do not improve expression, and indeed, one way to prevent toxicity is to not produce the membrane protein at all. This is indicated by the recovery of both large and small colonies in the Miroux and Walker selection, of which only the small colonies are found to express the target protein. In other words, the selected phenotype, which is the ability to survive on selective media and inducing conditions, does not necessarily indicate the desired result, which is overexpression of the target protein. And so, the selection recovers cells expressing the target protein as well as cells that do not express the protein, and thus the selection is not efficient.

In another recent study, a library of genes were screened for their ability to improve expression of G-protein coupled receptor (GPCR) targets fused to green fluorescent protein (GFP) in E. coli; host cell lines with improved expression due to coexpression of a library gene were isolated using fluorescence activated cell sorting (FACS) (Link et al., 2008). In a similar study, this group also used FACS to screen an E. coli transposon insertion library for variants expressing high levels of a GPCR target fused to GFP (Skretas and Georgiou, 2008). These studies lend further evidence that it is possible to engineer expression hosts for improved production of difficult to express targets, in this case GPCRs.

The methods of Link et al. and Skretas and Georgiou are limiting in a few respects (2008). Firstly, the expression of many difficult to express targets may be too low for the fluorescence of a GFP fusion to be detectable by a FACS approach, even if the target is expressed at improved levels. Consequently, FACS may not be applicable to poorly expressed target proteins that are the most in need of improved expression. Second, the numbers of clones that may be screened by FACS are much lower than those that may be selected by plating on selective media; typical flow cytometers can screen 10⁷ to 10⁸ cells per day, whereas many orders of magnitude more cells may be tested by plating on selective media (Link et al., 2008). Also, the specialized instrumentation needed for FACS is very costly, FACS methods are technically challenging, and sample preparation for FACS is time consuming (Link et al., 2008). A further limitation is that core facilities offering FACS services are often not prepared or willing to handle microbial samples (Link et al., 2008).”

### SUMMARY OF THE INVENTION

Methods are provided to select for host cell mutants that have an improved ability to recombinantly express target proteins. In this method, the coding sequence of the membrane protein of interest is fused to a C-terminal selectable marker and expressed off of an expression plasmid in an expression host, so that the production of the selectable marker and survival of the host on selective media is linked to expression of the targeted membrane protein. Thus, mutant host cells with improved expression properties can be directly selected. As there can be many ways for mutations to provide drug resistance that have nothing to do with expression of the fused membrane protein, we employ a dual selection strategy in which the same membrane protein target is fused to one of two drug resistance markers on two separate plasmids. The probability of obtaining mutations that confer resistance to both drugs without increasing membrane protein expression is extremely low.

The method of this invention for selecting host cell mutants that have an improved ability to recombinantly express a target protein comprises the steps of: (a) preparing an expression system consisting essentially of a host cell transformed with an expression vector encoding an inducibly expressed target protein fused to a C-terminal selectable marker and also encoding a constitutively expressed selectable marker to maintain the plasmid in the cell; (b) culturing cells transformed with the expression plasmid under selective pressure to maintain the plasmid and in the presence of a mutagen in order to randomly mutagenize the genome of the transformed cells; (c) selecting viable mutant cells on solid media under selective pressure and inducing conditions, so that only cells producing relatively high amounts of the target protein fused to the C-terminal selectable marker will survive; (d) pooling these selected cells; (e) transforming these pooled, selected mutants with a second compatible expression vector inducibly expressing the same target protein fused to a third C-terminal selectable marker and also constitutively expressing a fourth selectable marker in order to maintain this second plasmid in host cells; and (f) selecting viable cells on solid media under selective pressure and with induction, so that only cells producing relatively high levels of the target protein/C-terminal selectable marker fusion from both selection plasmids will survive. FIG. 2 provides a schematic of this selection method.

The invention also provides a method for efficiently curing isolated cells of the plasmids used during the selection process, in which the plasmids are removed by in vivo digestion with a rare-cutting endonuclease. In this method, the selection plasmids are engineered to contain a restriction site recognized by a rare-cutting endonuclease and are digested in vivo by this rare-cutting endonuclease. The rare-cutting endonuclease is inducibly expressed from a third temperature-sensitive vector that is subsequently removed from cells by outgrowth at an elevated temperature. With this method, host cell lines are completely cured in only two days.

The steps in the curing method supplied by this invention are: (a) transformation of the curing plasmid to host cells harboring one or more plasmids which contain the rare-cutting restriction site; (b) outgrowing transformed cells at 30° C. on inducing solid media selective for maintenance of the curing plasmid, during which time the rare-cutting restriction endonuclease encoded on the curing plasmid will be expressed and will digest the other plasmids containing the rare-cutting restriction site; and (c) streaking a single colony from the previous step to single colonies on media without selection for plasmid maintenance and without induction and outgrowing at 42° C. to remove the temperature-sensitive curing plasmid, finally resulting in completely cured mutant cells. FIG. 3 provides a schematic of this curing method.

The invention also provides host cells which display an improved ability to overexpress target proteins, e.g. selected by the methods of the invention. The host cells that have been isolated using the above described method are useful for overexpression of difficult to express proteins.

Further embodiments of the invention relate to specific systems for selecting mutant strains of E. coli that expresses target proteins at higher levels.

In other embodiments of the invention, the selection method is applied to isolate mutant hosts that express particular classes of hard to express proteins, such as membrane proteins.

### BRIEF DESCRIPTION OF THE DRAWINGS

FIG. 1. Plasmids used in this invention, in their preferred embodiment. (A) The selection system plasmids in their preferred embodiment. The coding sequence of the target membrane protein is fused to two different selectable markers on two compatible plasmids, called pSEL1 and pSEL2. Both plasmids utilize the P_{BAD} promoter. The plasmid pSEL1 has a p15A origin of replication and employs mouse dihydrofolate reductase (mDHFR) as a C-terminal selectable marker fusion, which confers resistance to trimethoprim. pSEL1 utilizes chloramphenicol resistance as the selectable marker for plasmid maintenance. Plasmid pSEL2 has kanamycin resistance (KanRes) as the C-terminal selectable marker fusion and bears a colE1 origin of replication. pSEL2 utilizes ampicillin resistance as the selectable marker for plasmid maintenance. The improved mutant hosts provided by this invention were selected using the selection plasmids pSEL1 and pSEL2 encoding the Mycobacterium tuberculosis rhomboid protease Rv1337. (B) The curing plasmid in its preferred embodiment. The two plasmids used to select improved mutants are removed by in vivo digestion with the rare-cutting endonuclease. pSEL1 and pSEL2 contain the 22 bp I-CreI cut site. I-CreI is encoded on a pSC101 plasmid with tetracycline resistance and a temperature sensitive origin of replication, and this construct is called pCURE.

FIG. 2. Schematic representation of the selection method provided by this invention in its preferred embodiment. A. E. coli host cells transformed with pSEL1 are mutagenized. B. In the first selection, mutant host cells expressing high levels of the target protein from pSEL1 are selected on the drug trimethoprim. C. Selected, mutant host cells are pooled and then transformed with pSEL2. D. In the second selection, mutant host cells expressing high levels of the target protein from both pSEL1 and pSEL2 are selected on the drugs trimethoprim and kanamycin. Selected mutant cells should express the target protein at higher levels than the wild type cell line.

FIG. 3. Schematic representation of the curing method provided by this invention in its preferred embodiment. On day 1, cells are transformed with pCURE and are then grown on LB agar under conditions which induce the expression of I-CreI. The expressed I-CreI digests the plasmids pSEL1 and pSEL2. On the 2^{nd} day, colonies are streaked onto LB agar and are outgrown at 42° C., which results in the loss of the temperature-sensitive plasmid pCURE. After two days, the resulting colonies are cured of all plasmids.

FIG. 4. Effectiveness of the selection. TOP10 cells producing a well-expressed membrane protein, the glycerol facilitator GlpF from E. coli, in the selection plasmids pSEL1 and pSEL2 were compared to cells expressing the poorly expressed membrane protein, signal peptide peptidase (SPP) from Archaeoglobus fulgidus, in pSEL1 and pSEL2. Only cells expressing GlpF are able to survive in the presence of selecting drug.

FIG. 5. The Curing Method is Efficient. Mutant EXP-R1337-5 harboring pSEL1 and pSEL2 was subjected to the curing method provided by this invention. Nine colonies resulting from the curing procedure were sampled for complete loss of plasmid. This was done by replica streaking colonies on LB Agar plates in this order: LB agar+no drug, LB agar+ampicillin, LB agar+chloramphenicol, LB agar+tetracycline, and finally LB agar+no drug. The absence of growth in the presence of ampicillin, chloramphenicol, and tetracylcine for all nine mutant EXP-R1337-5 samples indicates that all samples have been completely cured of all plasmids using the curing method provided by this invention.

FIG. 6. Increase in expression of MTb rhomboid-Rv1337 in five selected EXP mutants.

(A) A western blot using antibody specific for the N-terminal 6×His tag of the membrane protein rhomboid-Rv1337 expressed in pSEL1 and pSEL2 in the wild type strain and in 5 EXP mutant strains. Samples were normalized based on OD₆₀₀. A protein concentration standard (a 2-fold dilution series from 1000 ng to 3.9 ng of purified 6×His-tagged biotin ligase) was also loaded on each gel to quantify the increase in expression. (B) The fold increase in expression of rhomboid-Rv1337 in pSEL1 and pSEL2. Fold increase was determined by densitometry and comparison to the protein concentration standard. For all charts, the fold increase is averaged over three trials and the standard deviation is reported.

FIG. 7. Expression performance without a fusion to a selectable marker. MTb rhomboid-Rv1337 was expressed as a fusion in both pSEL1 and pSEL2 or without a fusion in both pBAD/HisA and pBAD/HisA/p15A. Relative mass units (RMU) are the amounts of membrane protein expressed relative to wild type, which is arbitrarily set at 1.0. RMUs were determined by analysis of western blots targeting the N-terminal 6×His tag of MTb rhomboid-Rv1337 and comparison to the protein concentration standard.

FIG. 8. Expression with the T7 promoter. Strains were tested for their ability to express MTb rhomboid-Rv1337 fused to kanamycin resistance using the T7 promoter. T7 polymerase was introduced into the wild type strain and each of the 5 EXP mutants by lysogenization with A-DE3. The fold increase in expression was determined by analysis of western blots targeting the N-terminal 6-histidine tag of each membrane protein and the protein concentration standard.

FIG. 9. Analysis of membrane expression. Fractionation of the wild type and mutant strains expressing MTb rhomboid-Rv1337 in pSEL1 and pSEL2 was performed. To assess the effectiveness of the fractionation, cultures were spiked before lysis with cells expressing a membrane protein (E. coli GlpF), a soluble protein (MBP), or a protein directed to inclusion bodies (streptavidin). The insoluble (IB), soluble (Sol), and membrane (MF) fractions of each sample were analyzed by western blotting targeting the 6×His tag of each protein.

FIG. 10. Increase in expression of other membrane protein targets. Various membrane protein targets were expression tested in the 5 EXP mutants selected using MTb rhomboid-Rv1337. The fold increase in expression was determined by analysis of western blots targeting the N-terminal 6×His tag of each membrane protein and the protein concentration standard. The MTb targets Rv2746 and Rv2835 were expressed as fusions to kanamycin resistance in pSEL2. A second rhomboid protease from MTb (Rv0110) was expressed without a fusion. Rhomboid proteases from Methanococcus jannaschii (MJR) and Drosophila melanogaster (Rho 1) were expressed with an N-terminal fusion to SUMO. The standard deviation of the fold increase in expression of MTb Rv2835 from strain EXP-Rv1337-5 is 55.

FIG. 11. Analysis of plasmid copy number. Strains were retransformed with pSEL1 and pSEL2 encoding rhomboid-Rv1337 and were grown to mid-log phase. Samples were normalized based on OD₆₀₀ and were column purified along with HindIII-digested pUC19 plasmid as an internal purification control. The samples were then analyzed on an agarose gel.

### DETAILED DESCRIPTION OF THE INVENTION

The methods of Miroux and Walker (U.S. Pat. No. 6,361,966) for improved protein expression in bacterial host cells could be improved by directly linking selection to target protein expression. In the methods of the present invention the selection is directly linked to membrane protein expression and thus greatly improves the efficiency of selection and screening. Furthermore, selection not only allows selection of cells that produce the target protein, but allows for the selection of the best mutant hosts that express the highest levels of protein out of a population of mutants, something that is not possible in the methods of Miroux and Walker (U.S. Pat. No. 6,361,966). The methods of the present invention are also applicable to all protein targets, and are not dependent on the degree of toxicity caused by overexpression of the target protein of interest.

A further improvement offered by the methods of the invention is the development of a method to cure improved mutants of the plasmids used during the selection process. To “cure” a host cell line is to remove all plasmids from the host cell line. The current art does not supply a universally effective and efficient curing method. Current methods involve outgrowing cells in the absence of selective pressure for maintenance of the plasmid and possibly in the presence of a small molecule curing agent and waiting for a cured cell line to emerge (Hirota, 1960, Denap et al., 2004). The current art of curing could take anywhere from a few days to a month and, as a result, is unreliable and requires a long period of outgrowth, which is undesirable. This invention addresses and solves this problem by supplying a curing system which can be used to efficiently and reliably cure host cells in only two days.

The current invention also has advantages over the methods of Link et. al and Skretas and Georgiou (2008). Firstly, much greater numbers of host cells may be analyzed for improved expression using the selection methods of this invention when compared FACS, thus greatly improving the chances of isolating a rare improved mutant cell line and greatly improving the efficiency of the method. Also, rather than relying on GFP fluorescence as a read out for improved expression, which may not be detectable when used as a fusion to a very poorly expressing protein, our invention employs selectable markers. When using a selectable marker as a fusion to a target protein to detect expression, very poorly expressed proteins can be detected on selective media and differences in very low expression levels can be determined by plating on drug gradients. Finally, the isolation of improved mutant cells lines on selective media based on the expression of a selectable marker is technically very easy, efficient, and fast, without the need for costly specialized instrumentation.

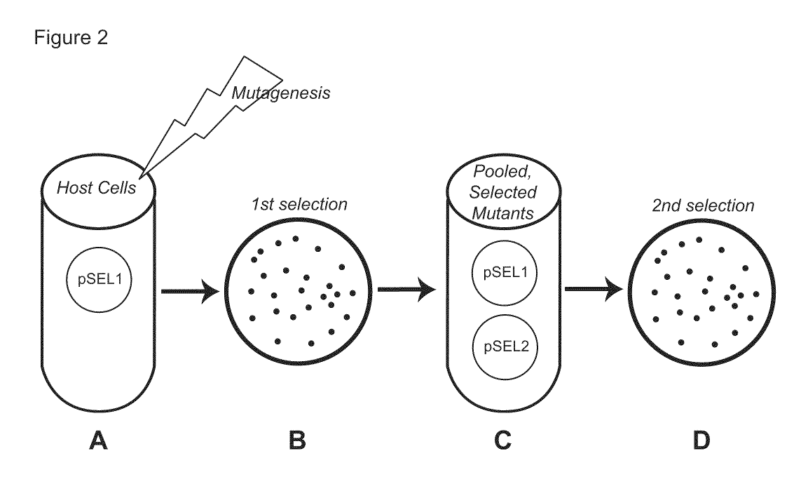

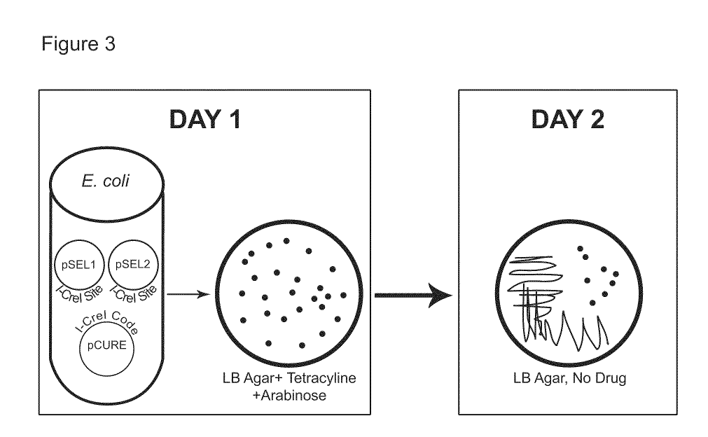

The present invention provides improvements over the current art by developing a simple, effective, and efficient strategy to select and cure mutants of E. coli that provide for higher level expression of toxic proteins, low-yielding proteins, and proteins that express as inclusion bodies. Another aspect of this invention is the production of improved mutants that have been isolated using the methods of this invention. These mutants increase expression of a target membrane protein at least about 5-fold, at least about 25-fold, at least about 75-fold, or more. A schematic representation of the selection method is provided in FIG. 2 and a schematic of the curing method is provided in FIG. 3.

As used herein, the term “determining” means to identify, i.e., establishing, ascertaining, evaluating, detecting or measuring, a value for a particular parameter of interest, e.g., bacterial growth, drug resistance, etc. The determination of the value may be qualitative (e.g., presence or absence) or quantitative, where a quantitative determination may be either relative (i.e., a value whose units are relative to a control (i.e., reference value) or absolute (e.g., where a number of actual molecules is determined).

The term “gene” as used herein is intended to refer to a nucleic acid sequence, which encodes a polypeptide. This definition includes various sequence polymorphisms, mutations, and/or sequence variants wherein such alterations do not affect the function of the gene product. The term “gene” is intended to include not only coding sequences but also regulatory regions such as promoters, enhancers, and termination regions. The term further includes all introns and other DNA sequences spliced from the mRNA transcript, along with variants resulting from alternative splice sites.

As used herein, the term “protein” means at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In certain embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood to one of ordinary skill in the art to which this invention belongs. Methods recited herein may be carried out in any order of the recited events that is logically possible, as well as the recited order of events.

All patents and other references cited in this application are incorporated into this application by reference except insofar as they may conflict with those of the present application (in which case the present application prevails). The citation of any publication is for its disclosure prior to the filing date and should not be construed as an admission that the present invention is not entitled to antedate such publication by virtue of prior invention.

### Plasmid Construction

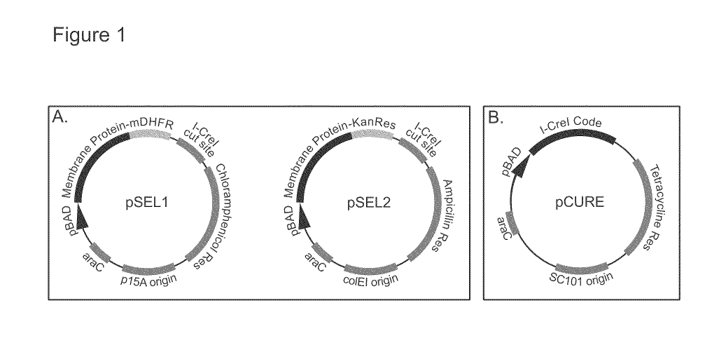

The plasmids used to select improved hosts are constructed using procedures known in the art. Herein, the term “selection plasmids” or the term “selection vectors” is used to describe the expression plasmids used in this invention to enable selection of hosts that are improved for overexpression of a target protein. In this method two selection plasmids are employed, and the preferred embodiment of the two selection plasmids is illustrated in FIG. 1A. Essentially, each selection plasmid encodes a target protein fused to a C-terminal selectable or screenable marker. Various components of these selection plasmids may be varied, as discussed below.

Plasmids, also known as vectors, for use in the invention may be constructed according to protocols known in the art, as provided, for example, in Sambrook and Russell (2001). cDNA or genomic DNA encoding a native or mutant target gene can be incorporated into vectors for manipulation. As used herein, “vector” refers to discrete elements that are used to introduce heterologous DNA into cells for expression, manipulation or replication thereof. Selection and use of such vehicles are well within the skill of the person of ordinary skill in the art. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Construction of vectors according to the invention may employ conventional ligation techniques or ligation-free techniques (Sambrook and Russell, 2001; Klock et al., 2008). Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing target gene expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridization, using an appropriately labeled probe based on a sequence provided herein, by analysis by polymerase chain reaction-based methods or by sequencing. Those skilled in the art will readily envisage how these methods may be modified, if desired.

An expression vector includes any vector capable of expressing target gene nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell results in expression of the cloned DNA. Especially preferred are episomal plasmid vectors for use in E. coli hosts, such as the pBAD vector which employs an arabinose induction system or a vector such as pET which employs the T7 polymerase expression system.

Expression and cloning vectors usually contain a promoter that is recognized by the host organism and is operably linked to target gene nucleic acid. Such a promoter may be constitutive or, more preferably, inducible. The promoters are operably linked to DNA encoding target gene by removing the promoter from the source DNA by restriction enzyme digestion and inserting the isolated promoter sequence into the vector. Both the native target gene promoter sequence and many heterologous promoters may be used to direct amplification and/or expression of target gene DNA.

Promoters suitable for use with prokaryotic hosts include, for example, the arabinose promoter system (Guzman et al., 1995), the T7 promoter system (Studier et at., 1990), the β-lactamase and lactose promoter systems, alkaline phosphatase, the tryptophan (trp) promoter system and hybrid promoters such as the tac promoter (De Boer et al., 1983). The promoter is generally either a promoter native to the microorganism (for example the E. coli arabinose promoter and the E. coli trpE promoter), a synthetic promoter such as the Tac promoter or a promoter obtainable from a heterologous organism, for example a virus, a bacterium or a bacteriophage such as phage λ or T7 which is capable of functioning in the microorganism. Their nucleotide sequences have been published, thereby enabling the skilled worker to operably ligate them to DNA encoding target gene, using linkers or adaptors to supply any required restriction sites. Promoters for use in bacterial systems will also generally contain a Shine-Delgarno sequence operably linked to the DNA encoding target gene. In the context of the present invention, the use of the arabinose promoter or the bacteriophage promoter T7 is particularly preferred.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the .alpha.- or a-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Both expression and cloning vectors generally contain nucleic acid sequences that enable the vector to replicate in one or more selected host cells. Typically, in cloning vectors, this sequence is one that enables the vector to replicate independently of the host chromosomal DNA, and includes origins of replication or autonomously replicating sequences. Vectors with different origins of replication thus avoid mutual exclusion during cell replication, that is to say, they are compatible plasmids. This is best achieved by using two different origins of replication, although other mechanisms, for example involving the use of two different selection markers, may also be used. Such sequences are well known for a variety of bacteria, yeast and viruses. The origin of replication from the plasmid pBR322 is suitable for most Gram-negative bacteria and the 2μ plasmid origin is suitable for yeast.

Most expression vectors are shuttle vectors, i.e. they are capable of replication in at least one class of organisms but can be transfected into another organism for expression. For example, a vector is cloned in E. coli and then the same vector is transfected into yeast cells even though it is not capable of replicating independently of the host cell chromosome. DNA may also be replicated by insertion into the host genome. However, the recovery of genomic DNA encoding target gene is more complex than that of exogenously replicated vector because restriction enzyme digestion is required to excise target gene DNA. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells when grown in a selective culture medium. Host cells not transformed with the vector containing the selection gene will not survive in the culture medium under selective conditions. Typical selection genes encode proteins that confer resistance to antibiotics and other toxins, e.g. ampicillin, chloramphenicol, kanamycin, trimethoprim, neomycin, methotrexate, or tetracycline, complement auxotrophic deficiencies, or supply critical nutrients not available from particular growth media.

As for a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

In this invention, selectable markers are used for two distinct purposes. In one function, a selectable marker is constitutively expressed from the plasmid independent of the expression of the target protein, and the function in this case is to maintain the plasmid in the host cell. In a second function, the selectable marker is a C-terminal fusion to the expressed target protein, and the function is to enable selection of host cells expressing the target protein at relatively high levels. The selectable markers used for these two purposes are usually different so that each can be selected for independently. Since there are two selection plasmids that are used in this invention, each with a selectable marker used for maintenance of the plasmid and a selectable marker used to enable selection for protein expression, there is usually a total of four independent selectable used in the method of selection of this invention. In the preferred embodiment, as illustrated in FIG. 1A, ampicillin- and chloramphenicol-resistance will be used to ensure maintenance of the two selection plasmids and kanamycin- and trimethoprim-resistance (DHFR) will be used as C-terminal fusions to the target protein to enable selection of improved expression hosts.

To increase the stringency of the selection, selectable markers known in the art can be mutated to decrease their effectiveness in granting survival. This would allow host cells expressing increasingly higher amounts of target protein fused to a C-terminal selectable marker to be selected, thus allowing the selection of even better mutant host cell lines.

In this invention it is preferable to use a selectable marker as a C-terminal fusion to isolate mutants improved for protein overexpression, but a screenable marker such as Green Fluorescent Protein (GFP) or any other reporter protein using color, fluorescence or antibody staining may be employed in order to identify and isolate mutant hosts improved for target protein expression. Where a fluorescent label is used, the cells may be directly screened on plates or in liquid culture or they may be selected by fluorescence activated cell sorting (FACS). Alternatively, antibody detection of a membrane protein targeted to the surface of a cell can be used to select for cells overexpressing the membrane protein using a FACS approach.

Target proteins cloned into the selection plasmids and thus used to select cells with an improved ability to express the target protein include membrane and globular proteins which are either foreign proteins or endogenous to the host. Particularly preferred are membrane proteins and examples are G-protein coupled receptors (GPCRs) and rhomboid proteases. These proteins have all been cloned and their sequences are readily available in the literature.

In the preferred embodiment of the selection method of this invention, the C-terminus of the target protein is found in the cytoplasm using selectable markers whose function is dependent on localization in the cytoplasm. If the C-terminus is periplasmic, a selectable marker such as β-lactamase (ampicillin resistance) or a screenable marker such as alkaline phosphatase (PhoA) could be utilized. Also, if the orientation of the target protein's C-terminus is not correctly oriented for use with the desired selectable marker, the C-terminus of the target protein could be engineered to have an opposite orientation.

In the preferred embodiment, the target protein and C-terminally linked selectable or screenable marker will be fused by an intermediate flexible linker. The linker may be variable in its amino acid composition and length. In the preferred embodiment it will be a threonine-serine-glycine sequence repeated four times to give a linker with the amino acid sequence (TSGTSGTSGTSG).

The vector will preferably include a nucleic acid sequence encoding a polypeptide which serves as a detectable label and/or the target gene itself may encode a detectable label. The detectable label gene may be placed in-frame with the target gene or may be a separate cistron in a di- or poly-cistronic operon with the target gene. This detectable label is useful in screening colonies in the final step of the process of the invention as it provides a rapid confirmation that colonies observed have retained the vector and express the target protein. The detectable label may be detected by western blotting or by ELISA. In the preferred embodiment, a 6-histidine tag will be included as a fusion to the expressed target protein and will be used to detect the presence of the expressed protein by western blotting.

The target gene according to the invention may include a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

In one embodiment, the two selection plasmids will be constructed as illustrated in FIG. 1A for use in E. coli TOP10 cells.

### Transformation of Selection Plasmids to Host Cells

The invention may be practiced employing any cells that can be grown in culture. Particularly preferred are bacterial and yeast hosts. Although the present invention is described with particular relevance to E. coli, other bacteria may also be used, in particular other members of the family Enterobacteriacae such as other members of the genera Escherichia or those of the genera Salmonella; Bacillaceae, such as Bacillus subtilis, Thermophilus and Lactobacillus; Pneumococcus; Streptococcus, and Haemophilus influenzae. Saccharomyces cerevisiae is a commonly used lower eukaryotic host microorganism. Others include Schizosaccharomyces pombe; Kluyveromyces sp.; Pichia pastoris; Candida; Neurospora crassa and Aspergillus hosts such as A. nidulans and A. niger.

Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognized when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (Sambrook and Russell, 2001).

Transfected or transformed cells are cultured using media and culturing methods known in the art. The composition of suitable media is known to those in the art, so that they can be readily prepared. Suitable culturing media are also commercially available.

Cultivation of the host cells may take place in the presence of selection pressure in order to maintain the vector, usually in the presence of an antibiotic which is metabolized by the selectable marker gene of the vector. The concentration of antibiotic used will depend upon the exact nature of the resistance gene and the concentration at which untransformed cells are killed by the antibiotic. In the case of ampicillin, somewhere between 20 and 200 μg per ml of culture will usually be sufficient, although this may be determined empirically if need be by those of skill in the art. In general, suitable concentrations of antibiotics may be determined by reference to standard laboratory reference books (e.g. Sambrook and Russell, 2001).

A preferred strain of E. coli is a K strain such as TOP10, JM109, or DK8, or a B strain such as BL21. These strains are widely available in the art from academic and/or commercial sources. The B strains are deficient in the Ion protease and other strains with this genotype may also be used. In the preferred embodiment, a K strain will be used so that transposition can be used in downstream mapping techniques. Most preferably the strain used is TOP10, which is available from Invitrogen, which is preferably used with the pBAD vector (Invitrogen) containing the arabinose promoter.

### Random Mutagenesis

In the method of this invention host cells may be randomly mutagenized through one of various means. One method is chemical mutagenesis by outgrowth in the presence of a mutagenizing reagent such as 2-aminopurine (2AP), N-methyl-N′-nitro-N-nitrosoguanidine (MNNG), or ethyl methane sulfonate (EMS), among others (Foster, 1991). Methods for chemical mutagenesis are well known in the art and are described in detail by Miller (1992). Mutagenesis may also be accomplished by expressing a mutator gene such as mutD5 off of a plasmid as described by Selifonova et al. (2001). Cellular genomes may also be manipulated through transposon mutagenesis, genome shuffling, overexpression of genes from a plasmid, or other cellular engineering techniques (Kleckner et I., 1991; Patnaik, 2008). Mutant cells may also be produced by simply outgrowing cells and allowing replication errors to naturally occur, as in the method of Miroux and Walker (U.S. Pat. No. 6,361,966).

In the preferred embodiment, cells harboring pSEL1 (FIG. 1A) will be mutagenized by expression of the mutator gene mutD5 off of a compatible plasmid with a temperature-sensitive origin of replication using methods described by Selifonova et al. (2001).

In order to obtain increasingly better mutants, 2 or more rounds of mutagenesis may be performed, and each round may use the same or a different method of mutagenesis.

### Two Step Selection

To select for expression we fuse the targeted membrane protein to a C-terminal selectable marker that confers a drug resistance phenotype, so that growth on selective media indicates expression of the target membrane protein. As there can be many ways for mutations to provide drug resistance that have nothing to do with expression of the fused membrane protein, we employ a dual selection strategy in which the same membrane protein target is fused to one of two drug resistance markers on two separate plasmids. The probability of obtaining mutations that confer resistance to both drugs without increasing membrane protein expression should be extremely low. Selecting with two selectable markers reduces the risk of false positives and greatly increases the probability that only cells which are indeed expressing the target protein at relatively high levels will be selected.

As illustrated in FIG. 2, the method of this invention for selecting host cell mutants that have an improved ability to recombinantly express a target protein comprises the steps of: (a) preparing an expression system consisting essentially of a host cell (E. coli TOP10 cells in the preferred embodiment) transformed with a selection vector (in the preferred embodiment pSEL1, as illustrated in FIG. 1A) encoding an inducibly expressed target protein fused to a C-terminal selectable marker and also encoding a constitutively expressed selectable marker to maintain the plasmid in the cell; (b) culturing cells transformed with the selection plasmid under selection pressure to maintain the plasmid in the presence of a mutagen (using mutD5 in the preferred embodiment) in order to randomly mutagenize the genome of the transformed cells; (c) selecting viable mutant cells on solid media under selective pressure (trimethoprim and chloramphenicol in the preferred embodiment) and inducing conditions, so that only cells producing relatively high amounts of the target protein fused to the C-terminal selectable marker will survive; (d) pooling these selected cells; (e) transforming these pooled, selected mutants with a second compatible selection vector (in the preferred embodiment pSEL2, as illustrated in FIG. 1A) which inducibly expresses the same target protein fused to a third C-terminal selectable marker and also constitutively expresses a fourth selectable marker in order to maintain this second plasmid in host cells; and (f) selecting viable cells on solid media under selective pressure (trimethoprim, chloramphenicol, kanamycin, and ampicillin in the preferred embodiment)and with induction, so that only cells expressing relatively high levels of the target protein/C-terminal selectable marker fusion from both selection plasmids will survive. The advantage of this two step procedure is that a vast number of mutagenized cells can be plated in the first step without losing mutants due to the low efficiency of plasmid transformation. The enriched pool from the first step can then be selected in the second step to isolate cells truly surviving due to expression of the target membrane protein. Also, by using this dual selection strategy the second plasmid never comes into contact with the mutagen.

### Screening Selected Mutants for Increased Expression

To confirm that isolated mutants are indeed improved for expression of the target protein when compared to the wild type host, various methods of screening may be performed. The expression vector may encode a polypeptide fusion to the target protein which serves as a detectable label or the target protein itself may serve as the selectable or screenable marker. The labeled protein may be detected via western blotting, ELISA, or, if the label is GFP, whole cell fluorescence or FACS. If the target protein expresses at sufficiently high levels, SDS PAGE may be performed to detect increases in mutant expression over wild type, in which case no label is necessary. In the preferred embodiment, a 6-histidine tag would be included as a fusion to the target protein, and this tag would be detected by western blotting.

### Plasmid Curing

It is necessary to cure selected mutants of the plasmids used in the selection process to ensure that the mutation responsible for the improvement in expression is in the genome of the host and not in the plasmid. Additionally, cell lines must be cured in order to apply the improved mutants to other protein targets and also to subject the mutants to additional rounds of mutagenesis.

Curing methods in the current art are in most cases inefficient and unreliable, which creates an obstacle to validation of the cell lines, further application of the cell lines, and further mutagenesis of the cell lines. Neither the current art nor the method of Miroux and Walker supplies a universally effective and efficient curing method. Current methods involve outgrowing cells in the absence of selective pressure for maintenance of the plasmid and possibly in the presence of a small molecule curing agent and subsequently waiting for a cured cell line to emerge (Hirota, 1960). The current art of curing could take anywhere from a few days to a month and, as a result, is unreliable and requires a long period of outgrowth, which is undesirable.

This invention includes an efficient method for curing host cell lines which can be accomplished in only two days. This curing method is illustrated in its preferred embodiment in FIG. 3. In the curing method of this invention, the two selection plasmids are digested with a rare-cutting restriction endonuclease. The two selection plasmids are engineered to contain a cut site recognized by a rare-cutting restriction. The rare-cutting restriction endonuclease is inducibly expressed off of a third plasmid, and this third plasmid is removed by outgrowth at an elevated temperature by virtue of its temperature sensitive origin of replication. This third plasmid, which we call the curing plasmid, can be constructed using traditional cloning methods (Sambrook and Russell, 2001). The third plasmid used in curing can contain any selectable marker or any origin of replication, but it is preferable that the selectable marker and origin of replication are different from those used in the two selection plasmids so that the curing plasmid can be maintained in the host cells and so that the curing plasmid can be independently selected for. The curing plasmid components may be interchanged as described above for the selection plasmids, particularly in terms of the plasmid used, the origin of replication used, the selectable marker used, and the promoter used. In the preferred embodiment, the origin of replication of the curing plasmid is preferably temperature sensitive so that the curing plasmid may be easily cured. The identity of the rare-cutting restriction endonuclease is not important, as long as it recognizes a restriction site that is sufficiently long so that cutting of the host's genome is very rare. Any of a few known homing endonucleases can be used to digest the two selection plasmids, and in the preferred embodiment I-CreI is used (Seligman et al., 1997). In the preferred embodiment, curing is effected by expression of the homing endonuclease I-CreI behind the inducible arabinose promoter off of a pSC101 plasmid with a temperature sensitive origin of replication and tetracycline resistance; this plasmid is called pCURE and is illustrated in FIG. 1B.

The curing method supplied by this invention is illustrated in its preferred embodiment in FIG. 3, and the steps are as follows: (a) transformation of the curing plasmid (in the preferred embodiment the curing plasmid pCURE is the curing plasmid) to host cells containing one or more plasmids which contain the rare-cutting restriction site (in the preferred embodiment, pSEL1 and pSEL2, which contain the I-CreI cut site); (b) outgrowing transformed cells at 30° C. on inducing solid media selective for maintenance of the curing plasmid, during which time the rare-cutting restriction endonuclease encoded on the curing plasmid will be expressed (I-CreI expressed from pCURE in the preferred embodiment) and will digest the other plasmids containing the rare-cutting restriction site (pSEL1 and pSEL2 in the preferred embodiment); and (c) streaking a single colony from the previous step to single colonies on media without selection for plasmid maintenance and without induction and outgrowing at 42° C. to remove the temperature-sensitive curing plasmid (pCURE in the preferred embodiment), finally resulting in completely cured host cells.

This curing method can be applied in any situation where expression of a target protein off of a plasmid is needed for only a short period of time.

Host Cells Obtained Using the Selection Method of this Invention

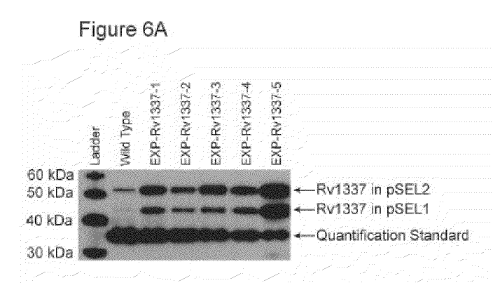

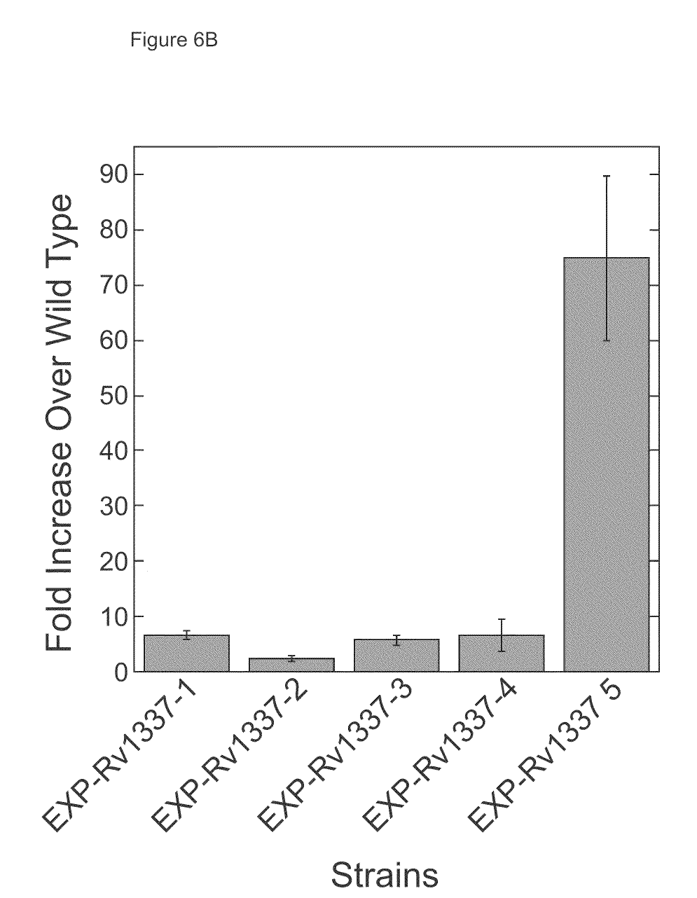

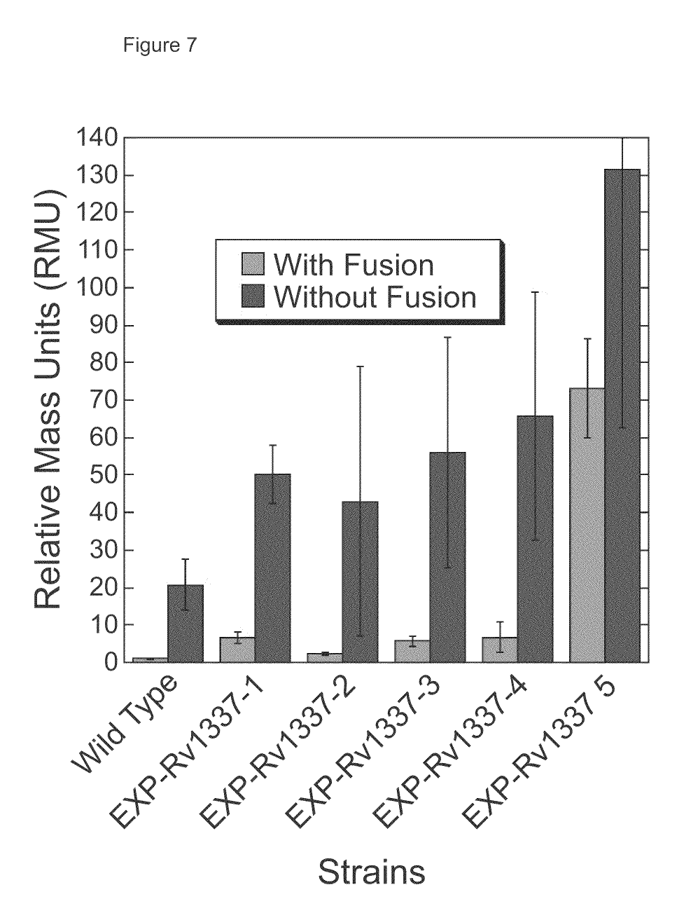

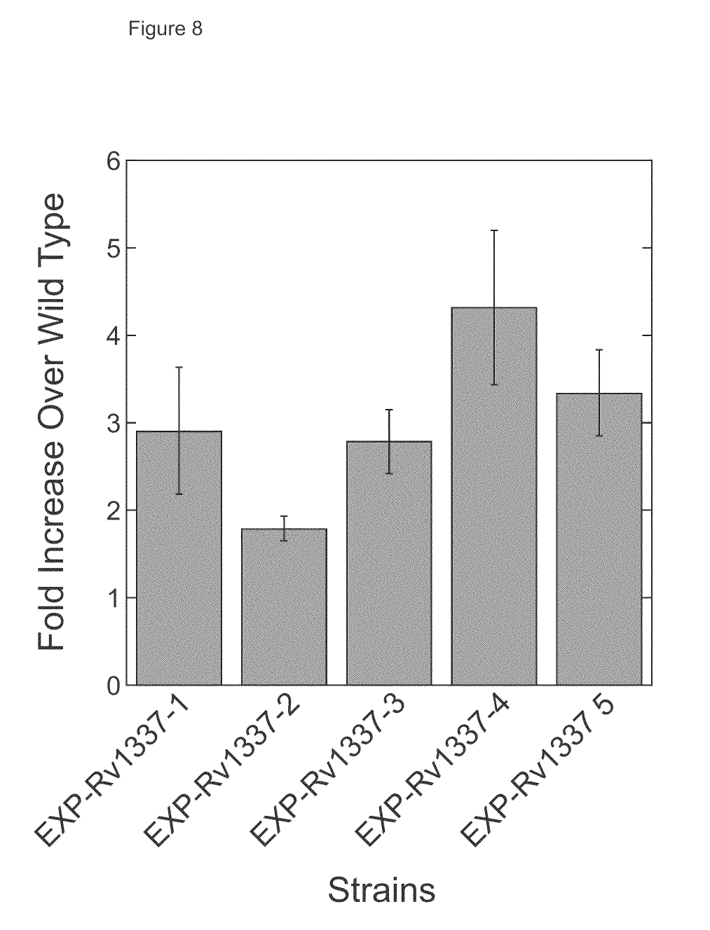

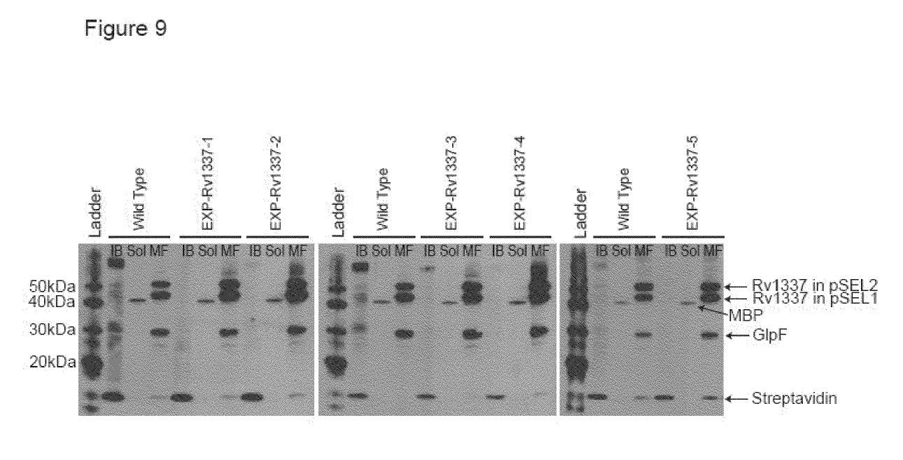

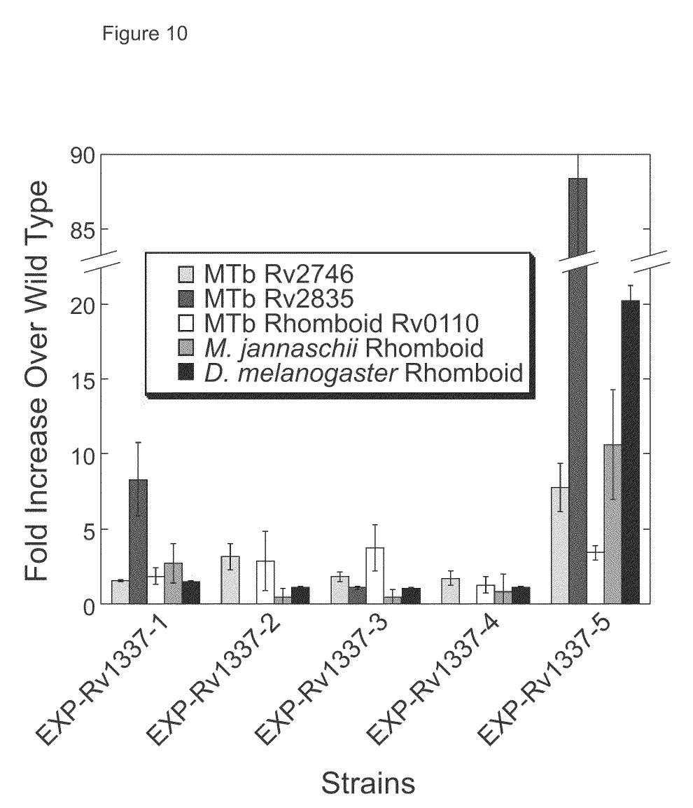

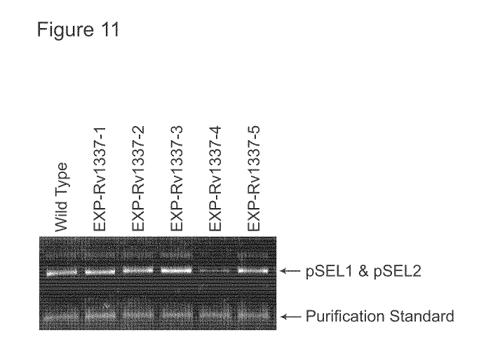

Host cells, preferably bacterial host cells, obtainable by any of the method of the invention, optionally cured of the vector, also form a further aspect of the invention. Particular bacteria include E. coli TOP10 mutants EXP-Rv1337-1, EXP-Rv1337-2, EXP-Rv1337-3, EXP-Rv1337-4, and EXP-Rv1337-5 which were isolated using the selection method detailed in this invention using the selection plasmids pSEL1 and pSEL2 encoding the rhomboid protease Rv1337 from Mycobacterium tuberculosis (MTb). These strains improve expression of MTb rhomboid-Rv1337 anywhere from 5- to 75-fold (FIG. 6). The EXP strains are effective for improving expression from both the T7 and the arabinose promoter (FIG. 8). These EXP strains, when cured, also provide a host for the expression of further proteins, especially low-expressing membrane proteins. We have shown that these mutants additionally improve expression of other membrane protein targets, including the Mycobacterium tuberculosis (MTb) targets Rv2746 and Rv2835 expressed as fusions to kanamycin, a second rhomboid protease from MTb (Rv0110) expressed without a fusion, and rhomboid proteases from Methanococcus jannaschii (MJR) and Drosophila melanogaster (Rho 1) expressed with an N-terminal fusion to SUMO (FIG. 10). The isolated mutant strains EXP-Rv1337-1, EXP-Rv1337-2, EXP-Rv1337-3, EXP-Rv1337-4, and EXP-Rv1337-5 may also be useful for expression of difficult to express globular proteins.

The Selection Method Indirectly Selects for Soluble Expression and/or Membrane Insertion

The method of selection used in this invention indirectly selects for soluble expression of the target protein and/or membrane insertion of the target protein rather than expression to inclusion bodies, as the fusion partner must be folded and active to confer drug resistance. We have observed that target protein expressed in the isolated mutant host cells EXP-Rv1337-1, EXP-Rv1337-2, EXP-Rv1337-3, EXP-Rv1337-4, and EXP-Rv1337-5 insert to the membrane, and these mutants in fact show a slight improvement over the wild type TOP10 strain in their ability to direct expressed protein to the membrane rather than to inclusion bodies (FIG. 9). And so, this method may produce strains which not only have an improved ability to overexpress target protein, but also have an improved ability to solubly express target proteins and/or effect membrane insertion of target membrane proteins.

### The Mutations in the EXP Mutants Responsible for Improved Expression can be Mapped

Some EXP strains are more effective for particular membrane proteins than others, suggesting that the mutants act by distinct mechanisms. This is also indicated by our finding that EXP-Rv1337-4 dramatically reduces plasmid copy number, while none of the other EXP strains display this phenotype (FIG. 11). It should in principle be possible to map the relevant mutations in the EXP mutants, and the identified genes may not only provide information about mechanism but will also enable more focused mutagenesis to achieve additional increases in expression. Furthermore, the discovered mutations can be combined in one strain to improve expression to an even greater degree.

### Examples

### Abbreviations Used

MTb, Mycobacterium tuberculosis; GlpF, glycerol facilitator protein; SPP, signal peptide peptidase.

### Example 1

### The Selection System is Effective

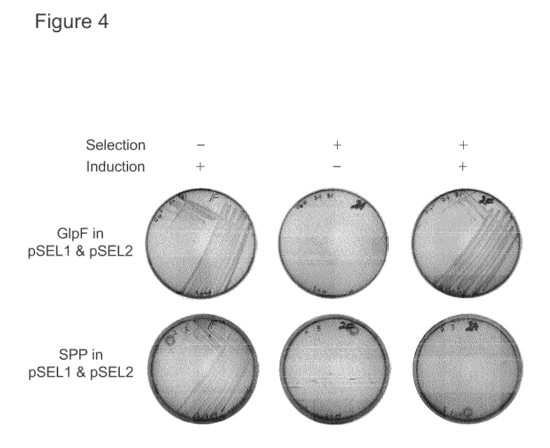

We initially tested the selection system provided by this invention by comparing growth on selective media of cells producing a well expressed membrane protein (GlpF from E. coli) and those expressing a poorly expressed membrane protein (SPP from Archaeoglobus fulgidus). In this example, GlpF and SPP were expressed using the plasmids pSEL1 and pSEL2, which are illustrated in FIG. 1A. As shown in FIG. 4, cells harboring GlpF in pSEL1 and pSEL2 and those harboring SPP in pSEL1 and pSEL2 both survived on inducing media without drugs, indicating that induction of neither protein is lethal to cells. In the presence of selecting drugs without induction, none of the constructs allowed survival. Under inducing conditions and in the presence of selection, only cells expressing the GlpF fusions survived. Thus, our selection system effectively and cleanly discriminated between cells expressing high levels of target membrane protein and those expressing little to no protein.

### Example 2

### Curing of Selected Mutants

After mutant selection, it is necessary to remove the selection plasmids from the strains.

We have found, however, that traditional curing methods were highly inefficient when applied to the strains and plasmids used in our work. We therefore developed a rapid and efficient curing method, which is provided by this invention and which is illustrated in FIG. 3.

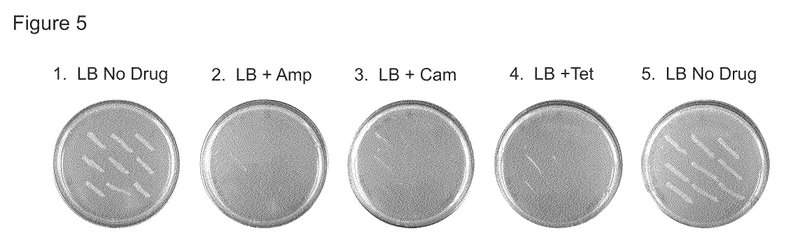

In the curing method of this invention, the plasmids used during selection are eliminated by in vivo digestion with a rare-cutting endonuclease, which in the preferred embodiment is the homing endonuclease I-CreI (Seligman et al. 1997). As shown in FIG. 1A, the recognition site for I-CreI was introduced into pSEL1 and pSEL2, the selection plasmids in the preferred embodiment of this invention. To remove the selection plasmids, we introduced a third plasmid, which we refer to as the curing plasmid, which encodes a rare-cutting endonuclease and preferably contains a temperature sensitive origin of replication The curing plasmid in its preferred embodiment is illustrated in FIG. 1B, and is called pCURE. pCURE encodes the homing endonuclease I-CreI, contains a temperature sensitive pSC101 origin of replication, and has tetracycline resistance as the selectable marker for plasmid maintenance. As illustrated in FIG. 3, I-CreI endonuclease expressed from pCURE digests the two pSEL plasmids, and pCURE is subsequently removed by growth at an elevated temperature (42° C.). Using the curing method provided by this invention strains can reliably be cured in only two days. FIG. 5 shows nine samples of mutant EXP-R1337-5 that have been completely cured using the curing method provided by this invention.

### Example 3

### Selection of Strains that Improve Expression of the Target Protein Rhomboid-Rv1337 from Mycobacterium tuberculosis (MTb)

With an effective selection system and a highly efficient curing system, we tested our ability to isolate E. coli mutants that improve membrane protein expression. We targeted the MTb alpha-helical inner membrane protein Rv1337, a rhomboid family protein, because it is a relatively large protein known from prior work to be expressed at low levels detectable by western blotting. In addition, rhomboid-Rv1337 has a cytoplasmic C-terminus, which is necessary for selection with the C-terminal selectable marker fusions used in pSEL1 and pSEL2.

Selection was performed in two steps as illustrated in FIG. 2. First, TOP10 cells harboring pSEL1 encoding rhomboid-Rv1337 were mutagenized with either the base analog 2-aminopurine (2AP) or the mutator gene mutD5, and colonies were selected for their ability to grow on media containing the drug trimethoprim. In the second step, the trimethoprim-resistant colonies were pooled and transformed with the pSEL2 construct encoding rhomboid-Rv1337. The cells, now harboring two plasmid constructs, were then selected on media containing both trimethoprim and kanamycin. The advantage of this two step procedure is that a vast number of mutagenized cells can be plated in the first step without losing mutants due to the low efficiency of plasmid transformation. The enriched pool can then be selected in the second step to isolate cells truly surviving due to expression of the target membrane protein. About 1 in 10,000 mutagenized cells survived the first trimethoprim selection (100-200 μg/mL), while roughly 1 in 1,000 of the trimethoprim-selected colonies survived the second step and were able to grow on both kanamycin (10-80 μg/mL) and trimethoprim (100-200 μg/mL).

We screened 47 selected colonies, and based on western blotting, 17 demonstrated increased expression of MTb rhomboid-Rv1337. We chose 5 clones, all from independently mutagenized cultures, that showed the greatest increase in protein production, and we cured them of the selection plasmids using the curing method described above. We refer to these strains as EXP-Rv1337-1, EXP-Rv1337-2, EXP-Rv1337-3, EXP-Rv1337-4, and EXP-Rv1337-5. These improved strains are a further aspect provided by this invention.

To validate the expression results and to show that the mutation is in the host and not the plasmid, we retransformed the cured mutants with pSEL1 and pSEL2 encoding MTb rhomboid-Rv1337. The increase in expression in the five selected, cured, and retransformed mutants is shown in FIG. 6. Qualitative examination of the western blot shown in FIG. 6A shows a clear enhancement of targeted membrane protein production. This increase was quantified by comparison to the intensities of known amounts of an added protein standard to the lanes. As shown in FIG. 6B, the selected strains improved expression of the MTb rhomboid-Rv1337 fusions up to 75-fold.

### Example 4

### Expression Without a Fusion to a Selectable Marker

As our general selection system requires the attachment of the target protein to a fusion partner, we wanted to test whether the mutations were effective when the protein was expressed without a marker protein attached. FIG. 7 charts expression of MTb rhomboid-Rv1337 either with or without fusions to selectable markers. For the wild type and all the mutant strains, the levels of expression with the fusion were clearly lower than expression without the fusion (FIG. 7). Nevertheless, the EXP strains improved expression of MTb rhomboid-Rv1337 when compared to wild type whether fused to a marker protein or not.

### Example 5

### Performance of Selected Strains with a T7 Promoter

We were interested to see if the mutant effects provided by this invention were specific to the arabinose promoter system we used in our selection process or if they were more generally effective. For example, the 041(DE3) and C43(DE3) effects seem to be specific to the T7 promoter (Miroux and Walker 1996, U.S. Pat. No. 6,361,966). We therefore lysogenized all 5 EXP mutant strains with ADE3 to introduce T7 RNA polymerase and tested the mutants for efficacy in a T7 promoter system. As shown in FIG. 8, all of the mutants improved expression of MTb rhomboid-Rv1337 behind the T7 promoter up to 4-fold. Thus, improved expression seen in the mutants is not completely specific to any one promoter type, although EXP-Rv1337-5 seems more effective for expression from an arabinose promoter.

### Example 6

### EXP Mutants Deliver Protein to the Membrane

We expected that the selection method provided by this invention would indirectly select for insertion into the membrane rather than inclusion bodies, as the fusion partner must be folded and active to confer drug resistance. To evaluate if the increased expression in the mutants corresponded to increased expression to the membrane we performed differential centrifugation. The insoluble, soluble, and membrane fractions of each sample were isolated and subsequently analyzed by western blotting targeting the 6-histidine tag of the expressed target protein, MTb rhomboid-Rv1337. Marker proteins for the various fractions were also included during the purification: (1) streptavidin, which is found in inclusion bodies, (2) maltose binding protein (MBP), which remains soluble and (3) GlpF, a protein targeted to the membrane. Each of these marker proteins has a 6-histidine tag. The results are shown in FIG. 9. In the wild type strain, rhomboid-Rv1337 expressed almost completely to the membrane, with a small amount of protein detected in the insoluble fraction. In all of the mutant strains, however, MTb rhomboid-Rv1337 appeared to be expressed exclusively to the membrane with no detectable component in the insoluble fraction. The EXP mutants, therefore, lead to insertion of the protein into the membrane rather than shunt the protein into inclusion bodies, and in fact, insertion seems to be improved in the selected mutants.

### Example 7

### Application of Selected Mutants to other Membrane Protein Targets

Although we selected for improved expression of MTb rhomboid-Rv1337, it is possible that the isolated strains provided by this invention could improve expression of other membrane proteins. To test this possibility, we expressed other MTb targets and a number of rhomboid constructs from various species in the wild type and all 5 EXP mutant strains (Table 1). As shown in FIG. 10, the expression of these targets could be improved in one or more of the selected mutants. EXP-Rv1337-5 is particularly effective for M. jannaschii rhomboid, D. melanogaster rhomboid, and MTb Rv2835, improving expression approximately 10, 20 and 90-fold, respectively.

### Example 8

### Relative Plasmid Copy Number of Mutants

We found that the expression of MTb rhomboid-Rv1337 fused to a selectable marker was improved when expressed from a single plasmid rather than two (not shown). Thus, one obvious way to increase expression would be to decrease plasmid copy number. We therefore evaluated the copy number of the 5 EXP mutants. As indicated in FIG. 11, all mutants had a plasmid copy number comparable to the wild type TOP10 strain, except for EXP-Rv1337-4 which showed a dramatically decreased copy number. It is likely that the reduced copy number in EXP-Rv1337-4 slows down expression to a level that the cells can more readily accommodate much like the C41 and C43 strains do for expression from the T7 promoter (Miroux and Walker, 1996, U.S. Pat. No. 6,361,966).

TABLE 1
Membrane protein targets expression tested in EXP mutants

| | GI | | | | # TM |
|---|---|---|---|---|---|
| Target | number | Organism | Putative Function | MW (kDa) | helices |
| Rv1337 | 15608477 | M. tuberculosis | rhomboid family protein | 25.8 | 6 |
| Rv2746 | 15609883 | M. tuberculosis | PGP synthase | 22.1 | 4 |
| Rv2835 | 15609972 | M. tuberculosis | ABC transporter | 34.1 | 6 |
| Rv0110 | 15607252 | M. tuberculosis | rhomboid family protein | 26.9 | 7 |
| MJR | 15669882 | M. jannashii | rhomboid family protein | 21.3 | 6 |
| Rho 1 | 24655197 | D. melanogaster | rhomboid family protein | 39.3 | 7 |

### REFERENCES

de Boer, H. A., Comstock, L. J., and Vasser, M. 1983. The tac promoter: a functional hybrid derived from the trp and lac promoters. Proc Natl Acad Sci USA 80: 21-25.
Denap, J. C., Thomas, J. R., Musk, D. J., and Hergenrother, P. J. 2004. Combating drug-resistant bacteria: small molecule mimics of plasmid incompatibility as antiplasmid compounds. J Am Chem Soc 126: 15402-15404.
Farid, S. S. 2007. Process economics of industrial monoclonal antibody manufacture. J Chromatogr B Analyt Technol Biomed Life Sci 848: 8-18.
Foster, P. L. 1991. In vivo mutagenesis. Methods Enzymol 204: 114-125.
Graumann, K., and Premstaller, A. 2006. Manufacturing of recombinant therapeutic proteins in microbial systems. Biotechnol J 1: 164-186.
Grisshammer, R. 2006. Understanding recombinant expression of membrane proteins. Curr Opin Biotechnol 17: 337-340.
Grisshammer, R., and Tate, C. G. 1995. Overexpression of integral membrane proteins for structural studies. Q Rev Biophys 28: 315-422.
Guzman, L. M., Belin, D., Carson, M. J., and Beckwith, J. 1995. Tight regulation, modulation, and high-level expression by vectors containing the arabinose PBAD promoter. J Bacteriol 177: 4121-4130.
Hirota, Y. 1960. The Effect of Acridine Dyes on Mating Type Factors in Escherichia Coli. Proc Natl Acad Sci USA 46: 57-64.
Hockney, R. C. 1994. Recent developments in heterologous protein production in Escherichia coli. Trends Biotechnol 12: 456-463.
Klammt, C., Schwarz, D., Eifler, N., Engel, A., Piehler, J., Haase, W., Hahn, S., Dotsch, V., and Bernhard, F. 2007. Reprint of “Cell-free production of G protein-coupled receptors for functional and structural studies” [J. Struct. Biol. 158 (2007) 482-493]. J Struct Biol 159: 194-205.
Kleckner, N., Bender, J., and Gottesman, S. 1991. Uses of transposons with emphasis on Tn10. Methods Enzymol 204: 139-180.
Klock, H. E., Koesema, E. J., Knuth, M. W., and Lesley, S. A. 2008. Combining the polymerase incomplete primer extension method for cloning and mutagenesis with microscreening to accelerate structural genomics efforts. Proteins 71: 982-994.
Korepanova, A., Gao, F. P., Hua, Y., Qin, H., Nakamoto, R. K., and Cross, T. A. 2005. Cloning and expression of multiple integral membrane proteins from Mycobacterium tuberculosis in Escherichia coli. Protein Sci 14: 148-158.
Lewinson, O., Lee, A. T., and Rees, D. C. 2008. The funnel approach to the precrystallization production of membrane proteins. J Mol Biol 377: 62-73.
Martinez Molina, D., Cornvik, T., Eshaghi, S., Haeggstrom, J. Z., Nordlund, P., and Sabet, M. I. 2008. Engineering membrane protein overproduction in Escherichia coli. Protein Sci 17: 673-680.
Link, A. J., Jeong, K. J., and Georgiou, G. 2007. Beyond toothpicks: new methods for isolating mutant bacteria. Nat Rev Microbiol 5: 680-688.
Link, A. J., Skretas, G., Strauch, E. M., Chari, N. S., and Georgiou, G. 2008. Efficient production of membrane-integrated and detergent-soluble G protein-coupled receptors in Escherichia coli. Protein Sci 17: 1857-1863.
Miller, J. H. 1992. A short course in bacterial genetics: a laboratory manual and handbook for Escherichia coli and related bacteria. Cold Spring Harbor Laboratory Press, Plainview, N.Y.
Miroux, B., and Walker, J. E. 1996. Over-production of proteins in Escherichia coli: mutant hosts that allow synthesis of some membrane proteins and globular proteins at high levels. J Mol Biol 260: 289-298.
Patnaik, R. 2008. Engineering complex phenotypes in industrial strains. Biotechnol Prog 24: 38-47.
Roosild, T. P., Greenwald, J., Vega, M., Castronovo, S., Riek, R., and Choe, S. 2005. NMR structure of Mistic, a membrane-integrating protein for membrane protein expression. Science 307: 1317-1321.
Sambrook, J., and Russell, D. W. 2001. Molecular cloning: a laboratory manual, 3rd ed. Cold Spring Harbor Laboratory Press, Cold Spring Harbor, N.Y.
Sarkar, C. A., Dodevski, I., Kenig, M., Dudli, S., Mohr, A., Hermans, E., and Pluckthun, A. 2008. Directed evolution of a G protein-coupled receptor for expression, stability, and binding selectivity. Proc Natl Acad Sci USA 105: 14808-14813.
Selifonova, O., Valle, F., and Schellenberger, V. 2001. Rapid evolution of novel traits in microorganisms. Appl Environ Microbiol67: 3645-3649.
Seligman, L. M., Stephens, K. M., Savage, J. H., and Monnat, R. J., Jr. 1997. Genetic analysis of the Chlamydomonas reinhardtii I-CreI mobile intron homing system in Escherichia coli. Genetics 147: 1653-1664.
Skretas, G., and Georgiou, G. 2008. Genetic analysis of G protein-coupled receptor expression in Escherichia coli: Inhibitory role of DnaJ on the membrane integration of the human central cannabinoid receptor. Biotechnol Bioeng.
Sorensen, H. P., and Mortensen, K. K. 2005. Advanced genetic strategies for recombinant protein expression in Escherichia coli. J Biotechnol 115: 113-128.
Studier, F. W., Rosenberg, A. H., Dunn, J. J., and Dubendorff, J. W. 1990. Use of T7 RNA polymerase to direct expression of cloned genes. Methods Enzymol 185: 60-89.
Terpe, K. 2006. Overview of bacterial expression systems for heterologous protein production: from molecular and biochemical fundamentals to commercial systems. Appl Microbiol Biotechnol 72: 211-222.
Wagner, S., Baars, L., Ytterberg, A. J., Klussmeier, A., Wagner, C. S., Nord, O., Nygren, P. A., van Wijk, K. J., and de Gier, J. W. 2007. Consequences of membrane protein overexpression in Escherichia coli. Mol Cell Proteomics 6: 1527-1550.
Wagner, S., Klepsch, M. M., Schlegel, S., Appel, A., Draheim, R., Tarry, M., Hogbom, M., van Wijk, K. J., Slotboom, D. J., Persson, J. O., et al. 2008. Tuning Escherichia coli for membrane protein overexpression. Proc Natl Acad Sci USA 105: 14371-14376.

What is claimed is:

1. A method for selecting host cell mutants having an improved ability to recombinantly express a target protein, the method comprising:
culturing host cells transformed with an expression vector encoding an inducibly expressed target protein fused to a C-terminal selectable or screenable marker, and encoding a constitutively expressed selectable marker to maintain the vector in the host cell, under selective pressure to maintain the plasmid and in the presence of a mutagen in order to randomly mutagenize the genome of the transformed cells;
selecting viable mutant cells under selective pressure and inducing conditions;
wherein said cells have an improved ability to recombinantly express said target protein.

2. The method of claim 1, wherein said selected mutants are subjected to a second round of selection, comprising the steps of:
pooling said selected cells;
transforming said pooled, selected cells with a second compatible expression vector inducibly expressing said target protein fused to a second C-terminal selectable or screenable marker and constitutively expressing a selectable marker to maintain the vector in the host cell; and
selecting viable cells under selective pressure and with induction;
wherein said cells have an improved ability to recombinantly express said target protein.”

3. The method of claim 1, wherein each of said vectors comprises a restriction site recognized by a rare-cutting endonuclease.

4. The method of claim 3, wherein said host cells are transformed or transfected with a vector encoding said rare-cutting endonuclease following said selection steps.

5. The method of claim 4, wherein said vector is temperature sensitive and inducibly expresses said rare-cutting endonuclease.

6. The method of claim 5, further comprising:
inducing expression of said rare-cutting endonuclease in order to effect plasmid curing following said selection steps; and
culturing said cells at an elevated temperature to remove said temperature sensitive vector.”

7. The method of claim 1, wherein said host cell is E. coli.

8. The method of claim 1, wherein the C-terminus of the target protein is found in the cytoplasm, and wherein said target protein is fused to a selectable markers whose function is dependent on localization in the cytoplasm.

9. The method of claim 1, wherein the C-terminus of the target protein is found in the periplasm.

10. The method of claim 1, wherein the target protein and C-terminally linked selectable or screenable marker are fused by an intermediate flexible linker.

11. The method of claim 10, wherein the linker has the amino acid sequence (TSGTSGTSGTSG).

12. The method of claim 1, wherein the vector comprises a nucleic acid sequence encoding a detectable label and/or the target gene encodes a detectable label.

13. The method of claim 1, wherein the target gene comprises a secretion sequence.

14. The method of claim 1, wherein the vector comprises a nucleic acid sequence encoding a detectable label and/or the target gene encodes a detectable label.

15. The method of claim 1, wherein the mutagen is selected from the group consisting of 2-aminopurine (2AP), N-methyl-N′-nitro-N-nitrosoguanidine (MNNG), ethyl methane sulfonate (EMS); and a mutator gene.

16. The method of claim 15, wherein the mutator gene is mutD5.

17. A host cell mutant obtained by the method of claim 1.

18. The host cell mutant of claim 17, wherein said cell is one of the E. coli TOP10 mutants EXP-Rv1337-1, EXP-Rv1337-2, EXP-Rv1337-3, EXP-Rv1337-4, or EXP-Rv1337-5.
